(19) United States
(12) Patent Application Publication
(10) Pub. No.: US 2017/0340484 A1
(43) Pub. Date: Nov. 30, 2017

(54) SYSTEM FOR PRODUCING A DEPICTION OF AN IMAGE

(71) Applicant: Shane Matthew Gillespie, Manistee, MI (US)

(72) Inventor: Shane Matthew Gillespie, Manistee, MI (US)

(21) Appl. No.: 15/165,313

(22) Filed: May 26, 2016

(51) Int. Cl. A61F 9/08; G02B 27/02; H04N 5/225; H04N 5/235

(52) U.S. Cl. CPC A61F 9/08; H04N 5/2354; G02B 27/022; H04N 5/2252; G06T 2207/10004

(57) ABSTRACT

A system for producing a depiction of an image is provided. The present invention includes camera operable to capture an image and a laser projector operable to project a laser beam in an x and y direction. The present invention further includes a computer having a microprocessor and a memory. A software may be saved on the memory and may operate the computer to scan the image to locate edges of objects depicted in the image and direct the laser projector to project the laser beam along x and y coordinates of a plane. The x and y coordinates outlines the edges of the objects and thereby forms the depiction of the image.

### BACKGROUND OF THE INVENTION

The present invention relates to producing depictions of images and, more particularly, to producing depictions of images using lasers.

Blind people have a limitation of not being able to see real world static and dynamic objects. Therefore they require other means of vision such as using a walking stick or having a guidance dog.

As can be seen, there is a need for devices that allow blind people to sense static and dynamic objects.

### SUMMARY OF THE INVENTION

In one aspect of the present invention, a system for producing a depiction of an image comprises: a camera operable to capture an image; a laser projector operable to project a laser beam in an x and y direction; and a computer comprising a microprocessor and a memory, wherein the microprocessor scans the image to locate edges of objects depicted in the image; and directs the laser projector to project the laser beam along x and y coordinates of a plane, wherein the x and y coordinates outline the edges of the objects and thereby form the depiction of the image.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention shown in use;

FIG. 2 is a rear perspective view of an embodiment of the present invention;

FIG. 3 is an exploded view of an embodiment of the present invention;

FIG. 4 is a section view of the present invention taken from 4-4 in FIG. 1;

FIG. 5 is a front view illustrating an exemplary 2d image captured by a camera of the present invention;

FIG. 6 is a schematic view of an outline projected onto a user by a laser beam;

FIG. 7 is a flow chart of an embodiment of the present invention;

FIG. 8 is a bottom view of an embodiment of a laser beam projector of the present invention;

FIG. 9 is a side view of an embodiment of a laser beam projector of the present invention;

FIG. 10 is a back view of an embodiment of a laser beam projector of the present invention;

FIG. 11 is a side view of an embodiment of a laser beam projector of the present invention; and

FIG. 12 is a section view of an embodiment of a laser beam projector of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a pseudo vision system for the blind. From a 2D digital spatial image manipulated by edge detection imaging software, a CW or pulsed laser is used with a 2D laser scanning system that can be made to communicate an altered image of edges to the blind persons through the sense of touch. An object range can also be communicated to the person through a histogram type graph under the 2D image of each object providing distances using ranging sensors, such as acoustic, LIDAR, or 3D cameras. This device then gives the blind person a pseudo 2D object range vision system and with little training, perceiving the 3D world is made possible.

The present invention provides a 2D touch sensation of images captured through the camera and object ranges. The present includes a laser spot size which is adjusted through the optical lens assembly unit 72. The scan will be calibrated from the laser spot size and the lasing time duration per pixel having a known image scan repetition frequency a calibration can be made to provide a safe operation. In certain embodiments, an absorbing gel may be applied and/or cooling fans to the persons back to provide a fast cooling response time for better nerve resolution.

The computer may run the main OS and have a program to operate the entire blind vision system. The camera may be attached to the computer. The imaging software is called through the blind vision system program to provide a binary image of edges in black and white fashion. The program then takes the image with edges and transfers out to the laser scanning system to scan a 2D image of the edges on the back skin area of the blind impaired, giving the blind impaired a 2D pseudo vision system and with intelligence and training can provide 3D capabilities. The laser scan may scan the image as a bitmap raster scan style or in another form such as an edge scan that slowly traces the edges. Another point would be laser control through PWM or timed pulses per location.

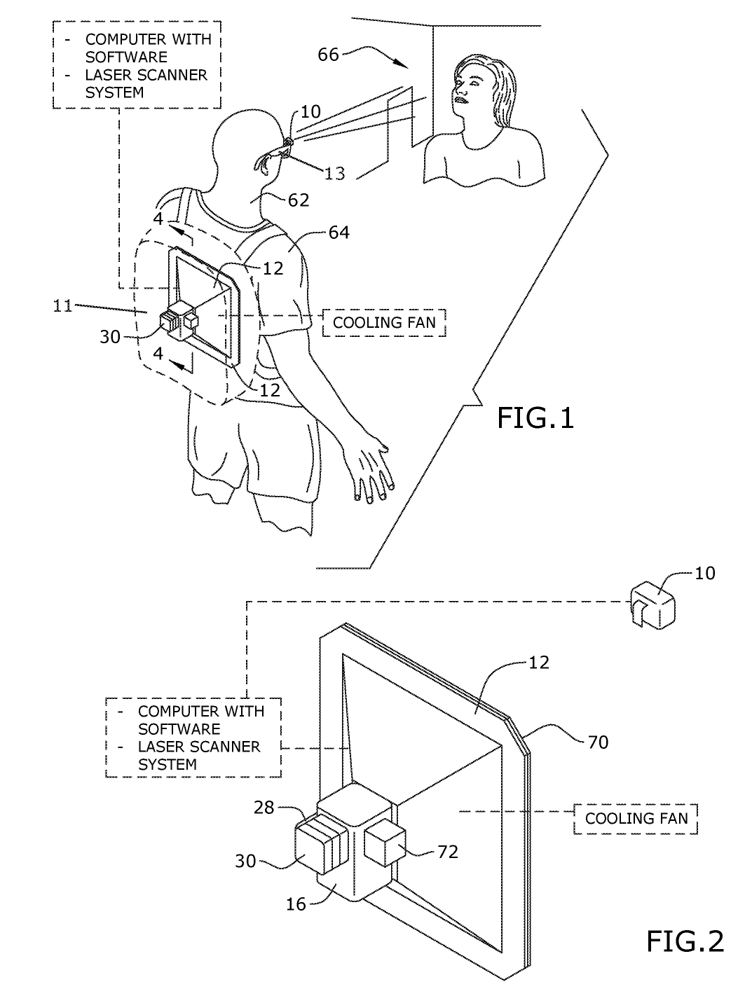

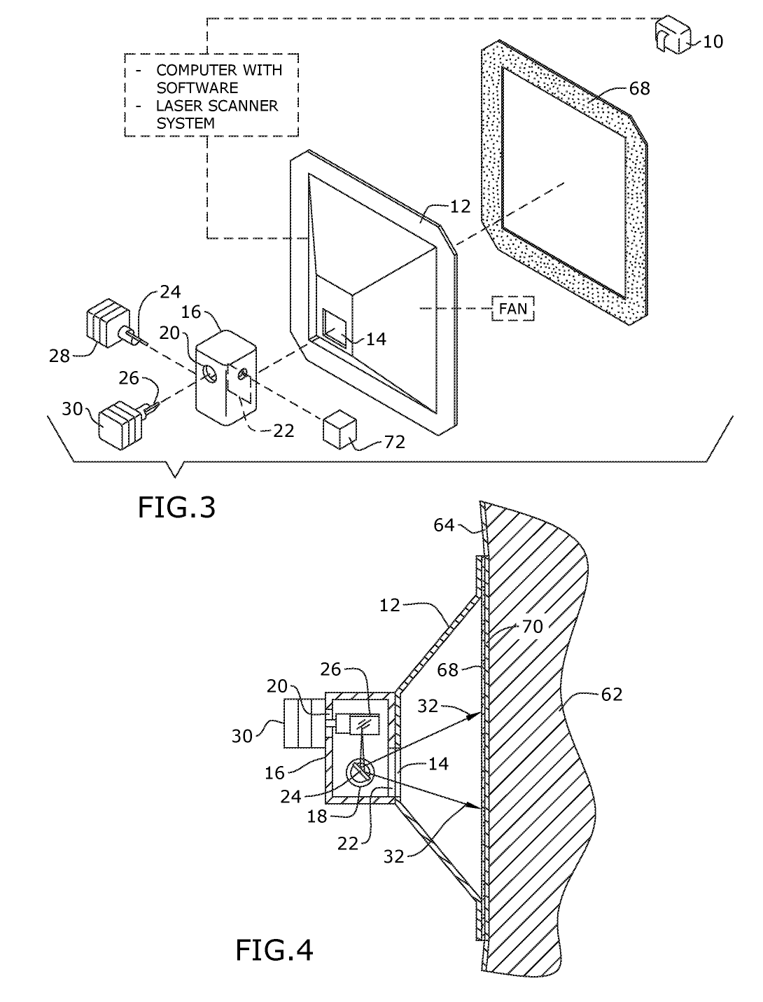

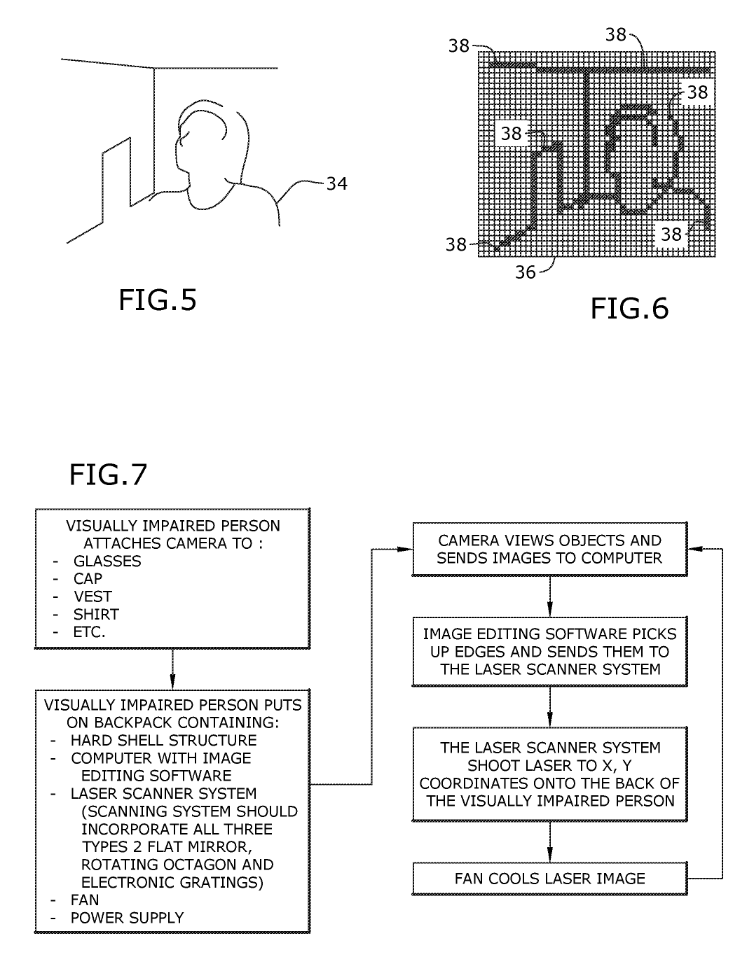

Referring to FIGS. 1 through 12, the present invention includes a system for producing a depiction of an image 34. The present invention includes camera 10 operable to capture an image 34 and a laser projector 16 operable to project a laser beam 32 in an x and y direction. The present invention further includes a computer having a microprocessor and a memory. Image editing software may be saved on the memory and may operate the computer to scan the image 34 to locate edges of objects 66 depicted in the image 34 and direct the laser projector to project the laser beam 32 along x and y coordinates 38 of a plane 36. The x and y coordinates 38 outlines the edges of the objects 66 and thereby forms the depiction of the image 34.

The present invention may further include a housing 12. The housing 12 forms an edge leading into the housing. The edge is within or adjacent to the plane 36. In certain embodiments, the housing 12 may include a pyramid shape having a flat top. The laser projector 16 may be secured to the flat top of the housing 12. In such embodiments, the laser projector 16 may include an opening 22 that aligns with an opening 14 formed through the flat top so that the laser beam 32 may travel through to the plane 36.

The housing 12 may be secured to the skin of a user 62. In such embodiments, the edge of the housing 12 may form a substantially flat flange. A foam padding 70 may be secured to the flange by an adhesive 68. The flange may fit in between an opening in the user's shirt 64 and the foam padding 70 may be pressed against the user's skin so that the laser beam 32 is projected onto the user's skin. In certain embodiments, the housing 12 is secured to a backpack 11. The housing 12 is positioned on the backpack 11 so that the laser beam 32 is projected on a user's back when wearing the backpack 11.

In certain embodiments, the present invention may include a cooling system to cool a user's skin. For example, a cooling fan may be disposed within the housing 12 and may be directed towards the user's skin. Alternatively, a cooling gel may be applied to a portion of the user's skin that receives the laser beam 32. The cooling system may provide a fast cooling response time for better nerve resolution.

The camera 10 of the present invention may be secured to an apparel 13. The apparel 13 is wearable by the user 62. The camera 10 is secured to the apparel 13 so that the camera 10 captures images 34 that are in front of the user 62 when the apparel is worn by the user 62. For example, the apparel 13 may include a pair of glasses, a shirt, a hat, a vest and the like. Therefore, a user may walk around and the laser projector 16 may emit the laser beam 32 on the user's skin depicting an image based on whatever the user is “looking” at.

The laser projector 16 of the present invention may include alternate configurations. In certain embodiments, the laser projector 16 includes a first mirror 26 rotatable about a first axis by a first motor 30 and a second mirror 24 rotatable about a second axis perpendicular to the first axis by a second motor 28. The mirrors 24, 26 may be rotated by shafts that fit through a first opening 20 and a second opening 18 formed through a housing. A laser beam emitter 72 emits the laser beam 32 to the first mirror 26, the first mirror 26 reflects the laser beam 32 to the second mirror 24 and the second mirror 24 reflects the laser beam 32 to the plane 36. The present invention may use servo motors for faster speeds.

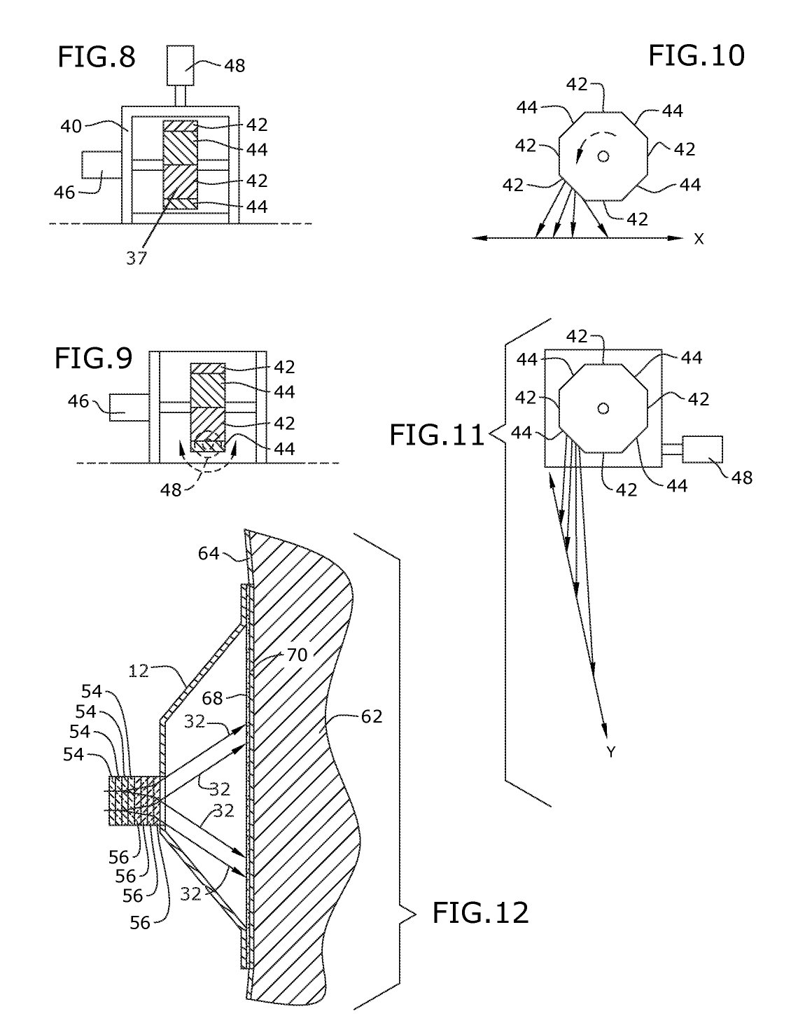

As illustrated in FIGS. 8 through 11, the laser projector 16 includes a reflector 37 having a prism shape, such as an octagonal prism shape. The reflector 37 includes a first base, a second base and a plurality of lateral faces. The plurality of lateral faces includes mirrors 42 and mirrors 44. The reflector 37 is secured within the reflector housing 40. A first motor 46 is operable to rotate the reflector 37 about a longitudinal axis running through the first base and the second base. A second motor 48 is operable to rotate the reflector housing 40 about a second axis perpendicular to the longitudinal axis, which rotates the reflector 37 about the second axis. The laser beam emitter 70 emits the laser beam 32 towards the plurality of lateral faces and the reflector 37 reflects the laser beam 32 to the plane 36.

As illustrated in FIGS. 12, the laser projector 16 may include a plurality of liquid crystal diffraction phase grating array x plates 54 and diffraction phase grating y plates 56. The plurality of liquid crystal diffraction phase grating array x plates 54 and diffraction phase grating y plates 56 are operable to receive the laser beam 32 and direct the laser beam 32 towards to the x and y coordinates of the plane 36. The transparent (laser travels through device) liquid crystal phase gratings are created by using the diffraction pattern from two circularly polarized UV lasers set at 90 degrees from each other, the liquid crystal device is then positioned at the intersection of these two lasers and a liquid crystal grating is formed, due to the UV curing resin contained within the liquid crystals. The liquid crystal diffraction phase grating can be produced as either a passive or active liquid crystal diffraction phase grating. The resulting liquid crystal diffraction phase grating device can then be stacked upon one another and together give a multitude of desired angle deflections (from varying grating thicknesses and electronically controlled gratings meaning beam deflection ON/OFF). The only difference between the x and y diffraction grating stack is that one is simply rotated ninety degrees from the other. These x and y liquid crystal diffraction phase grating stacks can be thought of as an electronically steered 2D scanning system, rather than the usual 2D mechanical laser beam steering methods.

The present invention may also include reflection phased arrays. Fine angle non-mechanical beam steering can be accomplished using Liquid Crystal on Silicon (LCoS) Optical Phased Array (OPA) devices. These reflective devices can use a unique backplane with a linear (one dimensional) array of tens of thousands of long thin electrodes. Again one set is rotated ninety-degrees from the other and the laser is reflected to the image plane.

It should be understood, of course, that the foregoing relates to exemplary embodiments of the invention and that modifications may be made without departing from the spirit and scope of the invention as set forth in the following claims.

What is claimed is:

1. A system for producing a depiction of an image comprising:
a camera operable to capture an image;
a laser projector operable to project a laser beam in an x and y direction; and
a computer comprising a microprocessor and a memory, wherein the microprocessor
scans the image to locate edges of objects depicted in the image; and
directs the laser projector to project the laser beam along x and y coordinates of a plane, wherein the x and y coordinates outline the edges of the objects and thereby form the depiction of the image.

2. The system of claim 1, further comprising a housing comprising an edge leading into the housing, wherein the edge is within or adjacent to the plane and the laser projector is secured to the housing.

3. The system of claim 2, wherein the housing comprises a pyramid shape comprising a top and a bottom, wherein the edge forms the bottom and the laser projector is secured to the top.

4. The system of claim 2, wherein the edge forms a flange, wherein a foam layer is adhered to the edge.

5. The system of claim 2, wherein the housing is secured to a backpack, wherein the housing is positioned on the backpack so that the laser beam is projected on a user's back when wearing the backpack.

6. The system of claim 1, wherein the laser projector comprises:
a first mirror rotatable about a first axis;
a second mirror rotatable about a second axis perpendicular to the first axis;
at least one motor operable to rotate the first mirror and the second mirror; and
a laser beam emitter, wherein
the laser beam emitter emits the laser beam to the first mirror, the first mirror reflects the laser beam to the second mirror and the second mirror reflects the laser beam to the plane.

7. The system of claim 1, wherein the laser projector comprises:
a reflector comprising a prism shape comprising a first base, a second base and a plurality of lateral faces, wherein each of the plurality of lateral faces comprises a mirror;
a reflector housing, wherein the reflector is secured within the reflector housing;
a first motor operable to rotate the reflector about a longitudinal axis running through the first base and the second base;
a second motor operable to rotate the reflector housing about a second axis perpendicular to the longitudinal axis, wherein the reflector rotates with the housing about the second axis; and
a laser beam emitter, wherein
the laser beam emitter emits a laser beam towards the plurality of lateral faces and the reflector reflects the laser beam to the plane.

8. The system of claim of claim 7, wherein the prism shape is an octagonal prism.

9. The system of claim 1, wherein the laser projector comprises:
a plurality of liquid crystal diffraction phase grating array x plates and iffraction phase grating y plates; and
a laser beam emitter, wherein the plurality of liquid crystal array diffraction phase grating x plates and diffraction phase grating y plates are operable to receive the laser beam and electronically direct the laser beam towards to the x and y coordinates of the plane.

10. The system of claim 1, further comprising an apparel wearable by a user, wherein the camera is secured to the apparel so that the camera captures the image in front of the user when worn by the user.

11. The system of claim 10, wherein the apparel is a pair of glasses.
